(19) United States
(12) Patent Application Publication
(10) Pub. No.: US 2006/0147966 A1
(43) Pub. Date: Jul. 6, 2006

(54) Preparation and labeling of polynucleotides for hybridization to a nucleic acid array

(75) Inventors: Anthony D. Barone, San Jose, CA (US); Glenn H. McGall, Palo Alto, CA (US)

Correspondence Address: AFFYMETRIX, INC;ATTN: CHIEF IP COUNSEL, LEGAL DEPT., 3420 CENTRAL EXPRESSWAY, SANTA CLARA, CA, 95051, US

(73) Assignee: Affymetrix, INC., Santa Clara, CA (US)

(21) Appl. No.: 11/314,034

(22) Filed: Dec. 20, 2005

Related U.S. Application Data
(60) Provisional application No. 60/640,481, filed on Dec. 30, 2004.

(51) Int. Cl. C12Q 1/68; C07H 21/04

(52) U.S. Cl. 435/6; 536/25.32

(57) ABSTRACT

In accordance with the present invention, method are presented for labeling a cDNA strand with a photochemical cleavable reagent which upon exposure to electromagnetic radiation of particular reagent to create abasic DNA sites. According to one aspect of the present invention, DNA at the abasic sites, also known a chemical lactone group, is cleaved with an endonuclease, for example an endonuclease IV, which cleaves the DNA and leaves a free 3′ OH group. This free 3′ OH group is then labeled with a terminal transferase to provide a detectable moiety. In accordance with a preferred aspect of the present invention,

### FIELD OF THE INVENTION

The present invention relates generally to the field of nucleic acid arrays. More specifically, the present invention relates to methods for cleaving and labeling DNA to prepare it for hybridization to a nucleic acid array.

### BACKGROUND OF THE INVENTION

Nucleic acid sample preparation and labeling methods have radically transformed laboratory research in the disciplines of genetics, molecular biology and recombinant DNA technology. Also impacted are fields as diverse as medical diagnostics, forensics, and gene expression monitoring, to name a few. There remains a need in the art for methods for reproducibly and efficiently fragmenting and labeling nucleic acids used for hybridization to oligonucleotide arrays.

### SUMMARY OF THE INVENTION

In one aspect of the invention, methods and compositions (including reagent kits) are provided for fragmenting nucleic acid samples. In preferred embodiments, the methods and compositions are used to fragment DNA samples for gene expression (transcript) monitoring and for genotyping assays. According to an aspect of the present invention, DNA is both fragmented and labeled via incorporation of photocleavable nucleotide derivatives. Photolysis of DNA strands bearing the derivatives results in elimination of the base (or base analog), leaving abasic sites. Chemically such sites may take the form of lactones. After creation of the abasic sites, the phophodiester backbone is susceptible to cleavage and labeling in a number of ways.

In a preferred embodiment, RNA transcript samples are used as templates for reverse transcription to synthesize single strand cDNA (ss-cDNA) or double strand cDNA (ds-cDNA). Methods for synthesizing cDNA are well known in the art. In another embodiment, the resulting cDNA may be used as a template for in vitro transcription reactions to synthesize cRNA. The cRNAs are then used as template for another cDNA synthesis reaction as described in Whole Transcript Assay (WTA) or small sample WTA (sWTA) protocols described for example in U.S. patent application Ser. No. 10/917,643.

In a preferred embodiment of the present invention, cDNA is synthesized in the presence of a photocleavable nucleotide derivative, wherein the incorporated photocleavable nucleotide derivative provides abasic DNA upon exposure to light of an appropriate wavelength; after exposing the cDNA to the appropriate wavelength of electromagnetic radiation, a plurality of abasic sites are created to provide abasic cDNA; according to one aspect of the present invention the abasic cDNA is cleaved with an endonuclease, such as Endonuclease IV, which generates a plurality of fragments with terminal free 3′ hydroxyl groups; such hydroxyl groups are substrates for the enzyme Terminal Transferase which can catalyze the formation of a phophodiester linkage with a nucleotide triphosphate or analogs thereof. Those of skill in the art are aware that Terminal Transferase will join a wide variety of triphosphate substrates to a free 3′ OH group at the terminus of a DNA strand. In accordance with an aspect of the present invention cDNA fragments are generated by photolysis are labeled with biotin using Terminal Transferase. Biotin labeled fragments are hybridized to a nucleic acid array to provide a hybridization pattern which then may be analyzed to determine the presence, absence or relative quantity of a particular fragment or gene. The most preferred biotin labeling reagent in accordance with the instant invention is DLR described in detail in U.S. patent Ser. No. 10/314,012, having the structure:

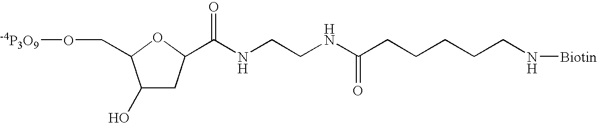

In another aspect of the present invention, creation of abasic DNA via photolysis is carried out as above. According to this aspect of the present invention, basic conditions, rather than an endonuclease, are used to cleave the DNA. However, this procedure leaves a phosphate on the 3′ —OH. In order to use Terminal Transferase to incorporate a label, the phosphate must be removed with, for example, and endonuclease.

In yet another aspect of the instant invention, abasic DNA is again generated by photolysis as described above. However, here there is no requirement for an independent cleavage step. Abasic DNA is reacted directly with a primary amine linked to a detectable moiety such as biotin. Preferably, the primary amine has the structure NH₂-L-Q, wherein Q is a detectable moiety and L is a linker.

Most preferably, the photocleavable nucleotide derivative is 3-Nitro-3-deaza-2′-deoxyadenosine triphosphate (NidA) having the structure:

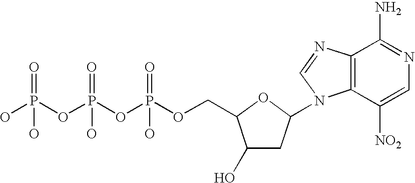

The fragmentation process produces DNA fragments within a certain range of length that can subsequently be labeled. In a preferred embodiment, the average size of fragments obtained is at least 10, 20, 30, 40, 50, 60, 70, 80, 100 or 200 nucleotides.

After fragments have been end-labeled, DNA fragments may be hybridized to a microarray of probes. Example of microarray that my be used for analysis are available from Affymetrix and include for example the HG-U133A2.0 array. In a preferred embodiment the arrays may have probes that target at least 50%, 60%, 70%, 80%, 90% or all the exons of at least 500, 1000 or 10000 transcripts.

The reagent kits of the invention typically include some combination of the reagents useful for the methods of the invention. For example, one reagent kit includes NidA, Endonuclease IV, DLR and a suitable microarray. Optionally, the reagent kit may include, for example, labeling reagents, reverse transcriptase, etc.

### DETAILED DESCRIPTION OF THE INVENTION

### A. GENERAL

The present invention has many preferred embodiments and relies on many patents, applications and other references for details known to those of the art. Therefore, when a patent, application, or other reference is cited or repeated below, it should be understood that it is incorporated by reference in its entirety for all purposes as well as for the proposition that is recited.

As used in this application, the singular form “a,” “an,” and “the” include plural references unless the context clearly dictates otherwise. For example, the term “an agent” includes a plurality of agents, including mixtures thereof.

An individual is not limited to a human being but may also be other organisms including but not limited to mammals, plants, bacteria, or cells derived from any of the above.

Throughout this disclosure, various aspects of this invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The practice of the present invention may employ, unless otherwise indicated, conventional techniques and descriptions of organic chemistry, polymer technology, molecular biology (including recombinant techniques), cell biology, biochemistry, and immunology, which are within the skill of the art. Such conventional techniques include polymer array synthesis, hybridization, ligation, and detection of hybridization using a label. Specific illustrations of suitable techniques can be had by reference to the example herein below. However, other equivalent conventional procedures can, of course, also be used. Such conventional techniques and descriptions can be found in standard laboratory manuals such as Genome Analysis: A Laboratory Manual Series (Vols. I-IV), Using Antibodies: A Laboratory Manual, Cells: A Laboratory Manual, PCR Primer: A Laboratory Manual, and Molecular Cloning: A Laboratory Manual (all from Cold Spring Harbor Laboratory Press), Stryer, L. (1995) Biochemistry (4th Ed.) Freeman, N.Y., Gait, “Oligonucleotide Synthesis: A Practical Approach” 1984, IRL Press, London, Nelson and Cox (2000), Lehninger, Principles of Biochemistry 3^{rd} Ed., W.H. Freeman Pub., New York, N.Y. and Berg et al. (2002) Biochemistry, 5^{th} Ed., W.H. Freeman Pub., New York, N.Y., all of which are herein incorporated in their entirety by reference for all purposes. The present invention can employ solid substrates, including arrays in some preferred embodiments. Methods and techniques applicable to polymer (including protein) array synthesis have been described in U.S. Ser. No. 09/536,841, WO 00/58516, U.S. Pat. Nos. 5,143,854, 5,242,974, 5,252,743, 5,324,633, 5,384,261, 5,405,783, 5,424,186, 5,451,683, 5,482,867, 5,491,074, 5,527,681, 5,550,215, 5,571,639, 5,578,832, 5,593,839, 5,599,695, 5,624,711, 5,631,734, 5,795,716, 5,831,070, 5,837,832, 5,856,101, 5,858,659, 5,936,324, 5,968,740, 5,974,164, 5,981,185, 5,981,956, 6,025,601, 6,033,860, 6,040,193, 6,090,555, 6,136,269, 6,269,846 and 6,428,752, in PCT Applications Nos. PCT/US99/00730 (International Publication Number WO 99/36760) and PCT/US01/04285, which are all incorporated herein by reference in their entirety for all purposes.

Nucleic acid arrays that are useful in the present invention include those that are commercially available from Affymetrix (Santa Clara, Calif.) under the brand name GeneChip®. Example arrays are shown on the website at affymetrix.com.

Patents that describe synthesis techniques in specific embodiments include U.S. Pat. Nos. 5,412,087, 6,147,205, 6,262,216, 6,310,189, 5,889,165, and 5,959,098. Nucleic acid arrays are described in many of the above patents, but the same techniques are applied to polypeptide arrays.

The present invention also contemplates many uses for polymers attached to solid substrates. These uses include gene expression monitoring, profiling, library screening, genotyping and diagnostics. Gene expression monitoring, and profiling methods can be shown in U.S. Pat. Nos. 5,800,992, 6,013,449, 6,020,135, 6,033,860, 6,040,138, 6,177,248 and 6,309,822. Genotyping and uses therefore are shown in U.S. Ser. No. 60/319,253, 10/013,598, and U.S. Pat. Nos. 5,856,092, 6,300,063, 5,858,659, 6,284,460, 6,361,947, 6,368,799 and 6,333,179. Other uses are embodied in U.S. Pat. Nos. 5,871,928, 5,902,723, 6,045,996, 5,541,061, and 6,197,506.

The present invention also contemplates sample preparation methods in certain preferred embodiments. Prior to or concurrent with genotyping, the genomic sample may be amplified by a variety of mechanisms, some of which may employ PCR. See, e.g., PCR Technology: Principles and Applications for DNA Amplification (Ed. H. A. Erlich, Freeman Press, NY, N.Y., 1992); PCR Protocols: A Guide to Methods and Applications (Eds. Innis, et al., Academic Press, San Diego, Calif., 1990); Mattila et al., Nucleic Acids Res. 19, 4967 (1991); Eckert et al., PCR Methods and Applications 1, 17 (1991); PCR (Eds. McPherson et al., IRL Press, Oxford); and U.S. Pat. Nos. 4,683,202, 4,683,195, 4,800,159 4,965,188,and 5,333,675, and each of which is incorporated herein by reference in their entireties for all purposes. The sample may be amplified on the array. See, for example, U.S. Pat. No 6,300,070 and U.S. patent application Ser. No. 09/513,300, which are incorporated herein by reference.

Other suitable amplification methods include the ligase chain reaction (LCR) (e.g., Wu and Wallace, Genomics 4, 560 (1989), Landegren et al., Science 241, 1077 (1988) and Barringer et al. Gene 89:117 (1990)), transcription amplification (Kwoh et al., Proc. Natl. Acad. Sci. USA 86, 1173 (1989) and WO88/10315), self-sustained sequence replication (Guatelli et al., Proc. Nat. Acad. Sci. USA, 87, 1874 (1990) and WO90/06995), selective amplification of target polynucleotide sequences (U.S. Pat. No 6,410,276), consensus sequence primed polymerase chain reaction (CP-PCR) (U.S. Pat. No 4,437,975), arbitrarily primed polymerase chain reaction (AP-PCR) (U.S. Pat. No 5,413,909, 5,861,245) and nucleic acid based sequence amplification (NABSA). (See, U.S. Pat. Nos. 5,409,818, 5,554,517, and 6,063,603, each of which is incorporated herein by reference). Other amplification methods that may be used are described in, U.S. Pat. Nos. 5,242,794, 5,494,810, 4,988,617 and in U.S. Ser. No. 09/854,317, each of which is incorporated herein by reference.

Additional methods of sample preparation and techniques for reducing the complexity of a nucleic sample are described in Dong et al., Genome Research 11, 1418 (2001), in U.S. Pat. No 6,361,947, 6,391,592 and U.S. patent application Ser. Nos. 09/916,135, 09/920,491, 09/910,292, and 10/013,598.

Methods for conducting polynucleotide hybridization assays have been well developed in the art. Hybridization assay procedures and conditions will vary depending on the application and are selected in accordance with the general binding methods known including those referred to in: Maniatis et al. Molecular Cloning: A Laboratory Manual (2^{nd} Ed. Cold Spring Harbor, N.Y, 1989); Berger and Kimmel Methods in Enzymology, Vol. 152, Guide to Molecular Cloning Techniques (Academic Press, Inc., San Diego, Calif., 1987); Young and Davism, P.N.A.S, 80: 1194 (1983). Methods and apparatus for carrying out repeated and controlled hybridization reactions have been described in U.S. Pat. Nos. 5,871,928, 5,874,219, 6,045,996 and 6,386,749, 6,391,623 each of which are incorporated herein by reference The present invention also contemplates signal detection of hybridization between ligands in certain preferred embodiments. See U.S. Pat. Nos. 5,143,854, 5,578,832; 5,631,734; 5,834,758; 5,936,324; 5,981,956; 6,025,601; 6,141,096; 6,185,030; 6,201,639; 6,218,803; and 6,225,625, in U.S. Patent application 60/364,731 and in PCT Application PCT/US99/06097 (published as WO99/47964), each of which also is hereby incorporated by reference in its entirety for all purposes.

Methods and apparatus for signal detection and processing of intensity data are disclosed in, for example, U.S. Pat. Nos. 5,143,854, 5,547,839, 5,578,832, 5,631,734, 5,800,992, 5,834,758; 5,856,092, 5,902,723, 5,936,324, 5,981,956, 6,025,601, 6,090,555, 6,141,096, 6,185,030, 6,201,639; 6,218,803; and 6,225,625, in U.S. Patent application 60/364,731 and in PCT Application PCT/US99/06097 (published as WO99/47964), each of which also is hereby incorporated by reference in its entirety for all purposes.

The practice of the present invention may also employ conventional biology methods, software and systems. Computer software products of the invention typically include computer readable medium having computer-executable instructions for performing the logic steps of the method of the invention. Suitable computer readable medium include floppy disk, CD-ROM/DVD/DVD-ROM, hard-disk drive, flash memory, ROM/RAM, magnetic tapes and etc. The computer executable instructions may be written in a suitable computer language or combination of several languages. Basic computational biology methods are described in, e.g. Setubal and Meidanis et al., Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics: Application in Biological Science and Medicine (CRC Press, London, 2000) and Ouelette and Bzevanis Bioinformatics: A Practical Guide for Analysis of Gene and Proteins (Wiley & Sons, Inc., 2^{nd} ed., 2001).

The present invention may also make use of various computer program products and software for a variety of purposes, such as probe design, management of data, analysis, and instrument operation. See, U.S. Pat. Nos. 5,593,839, 5,795,716, 5,733,729, 5,974,164, 6,066,454, 6,090,555, 6,185,561, 6,188,783, 6,223,127, 6,229,911 and 6,308,170.

Additionally, the present invention may have preferred embodiments that include methods for providing genetic information over networks such as the Internet as shown in U.S. patent applications Ser. Nos. 10/197,621, 10/063,559 (U.S. Publication No. 20020183936), Ser. Nos. 10/065,868, 10/328,818, 10/328,872, 10/423,40360/349,546, and 60/482,389.

### B. DEFINITIONS

The term “abasic site” refers to a nucleotide in a DNA strand wherein the base structure has been removed or extracted. In accordance with the present invention, it is preferred to create abasic sites via a photocleavable nucleotide derivative. This is shown below:

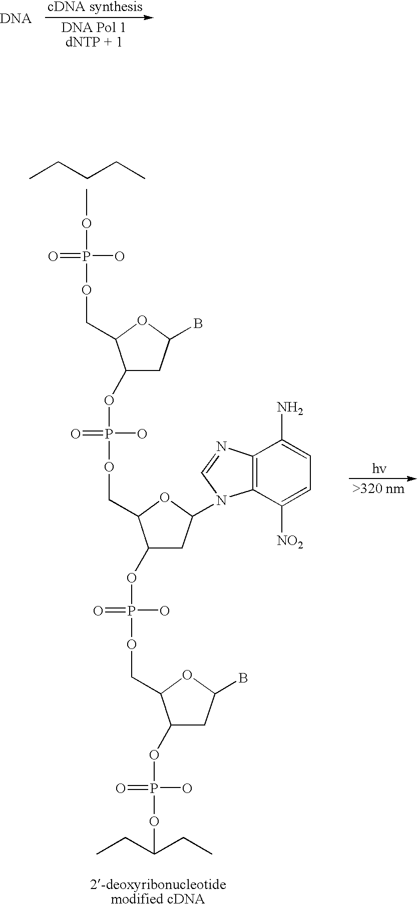

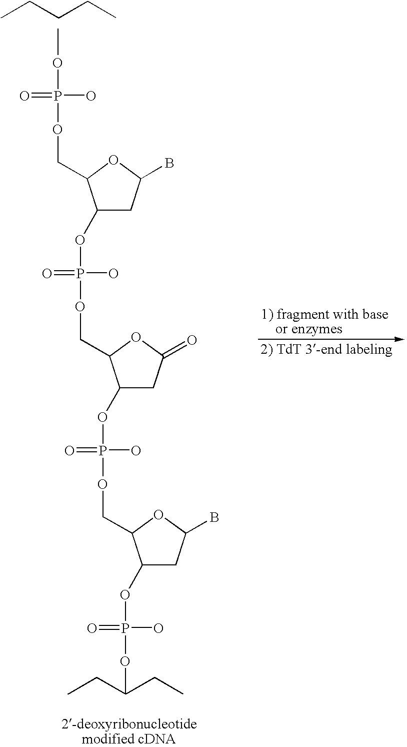

The term “array” as used herein refers to an intentionally created collection of molecules which can be prepared either synthetically or biosynthetically. The molecules in the array can be identical or different from each other. The array can assume a variety of formats, for example, libraries of soluble molecules; libraries of compounds tethered to resin beads, silica chips, or other solid supports.

The term “biotin” as used in the context of an aspect of the present invention generally refers to the moiety represented by the following formula: 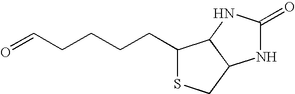
Molecules are generally shown in amide linkage to the biotin. Thus, for example, the DLR triphosphate molecule used to label '3 OH groups has the formula:

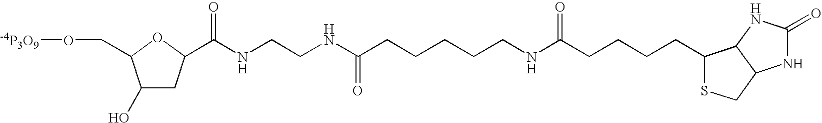

The term “complementary” as used herein refers to the hybridization or base pairing between nucleotides or nucleic acids, such as, for instance, between the two strands of a double stranded DNA molecule or between an oligonucleotide primer and a primer binding site on a single stranded nucleic acid to be sequenced or amplified. Complementary nucleotides are, generally, A and T (or A and U), or C and G. Two single stranded RNA or DNA molecules are said to be complementary when the nucleotides of one strand, optimally aligned and compared and with appropriate nucleotide insertions or deletions, pair with at least about 80% of the nucleotides of the other strand, usually at least about 90% to 95%, and more preferably from about 98 to 100%. Alternatively, complementarity exists when an RNA or DNA strand will hybridize under selective hybridization conditions to its complement. Typically, selective hybridization will occur when there is at least about 65% complementary over a stretch of at least 14 to 25 nucleotides, preferably at least about 75%, more preferably at least about 90% complementary. See, M. Kanehisa Nucleic Acids Res. 12:203 (1984), incorporated herein by reference.

The term “detectable moiety” (Q) means a chemical group that provides a signal. The signal is detectable by any suitable means, including spectroscopic, photochemical, biochemical, immunochemical, electrical, optical or chemical means. In certain cases, the signal is detectable by 2 or more means.

The detectable moiety provides the signal either directly or indirectly. A direct signal is produced where the labeling group spontaneously emits a signal, or generates a signal upon the introduction of a suitable stimulus. Radiolabels, such as ³H, ¹²⁵I, ³⁵S, ¹⁴C or ³²P, and magnetic particles, such as Dynabeads™, are nonlimiting examples of groups that directly and spontaneously provide a signal. Labeling groups that directly provide a signal in the presence of a stimulus include the following nonlimiting examples: colloidal gold (40-80 nm diameter), which scatters green light with high efficiency; fluorescent labels, such as fluorescein, Texas red, Rhoda mine, and green fluorescent protein (Molecular Probes, Eugene, Oreg.), which absorb and subsequently emit light; chemiluminescent or bioluminescent labels, such as luminol, lophine, acridine salts and luciferins, which are electronically excited as the result of a chemical or biological reaction and subsequently emit light; spin labels, such as vanadium, copper, iron, manganese and nitroxide free radicals, which are detected by electron spin resonance (ESR) spectroscopy; dyes, such as quinoline dyes, triarylmethane dyes and acridine dyes, which absorb specific wavelengths of light; and colored glass or plastic (e.g., polystyrene, polypropylene, latex, etc.) beads. See U.S. Pat. Nos. 3,817,837; 3,850,752; 3,939,350; 3,996,345; 4,277,437; 4,275,149 and 4,366,241.

A detectable moiety provides an indirect signal where it interacts with a second compound that spontaneously emits a signal, or generates a signal upon the introduction of a suitable stimulus. Biotin, for example, produces a signal by forming a conjugate with streptavidin, which is then detected. See Hybridization With Nucleic Acid Probes. In Laboratory Techniques in Biochemistry and Molecular Biology; Tijssen, P., Ed.; Elsevier: New York, 1993; Vol. 24. An enzyme, such as horseradish peroxidase or alkaline phosphatase, that is attached to an antibody in a label-antibody-antibody as in an ELISA assay, also produces an indirect signal.

A preferred detectable moiety is a fluorescent group. Fluorescent groups typically produce a high signal to noise ratio, thereby providing increased resolution and sensitivity in a detection procedure. Preferably, the fluorescent group absorbs light with a wavelength above about 300 nm, more preferably above about 350 nm, and most preferably above about 400 nm. The wavelength of the light emitted by the fluorescent group is preferably above about 310 nm, more preferably above about 360 nm, and most preferably above about 410 nm.

The fluorescent detectable moiety is selected from a variety of structural classes, including the following nonlimiting examples: 1- and 2-aminonaphthalene, p,p′diaminostilbenes, pyrenes, quaternary phenanthridine salts, 9-aminoacridines, p,p′-diaminobenzophenone imines, anthracenes, oxacarbocyanine, marocyanine, 3-aminoequilenin, perylene, bisbenzoxazole, bis-p-oxazolyl benzene, 1,2-benzophenazin, retinol, bis-3-aminopridinium salts, hellebrigenin, tetracycline, sterophenol, benzimidazolyl phenylamine, 2-oxo-3-chromen, indole, xanthen, 7-hydroxycoumarin, phenoxazine, salicylate, strophanthidin, porphyrins, triarylmethanes, flavin, xanthene dyes (e.g., fluorescein and rhodamine dyes); cyanine dyes; 4,4-difluoro-4-bora-3a,4a-diaza-s-indacene dyes and fluorescent proteins (e.g., green fluorescent protein, phycobiliprotein).

A number of fluorescent compounds are suitable for incorporation into the present invention. Nonlimiting examples of such compounds include the following: dansyl chloride; fluoresceins, such as 3,6-dihydroxy-9-phenylxanthhydrol; rhodamineisothiocyanate; N-phenyl-1-amino-8-sulfonatonaphthalene; N-phenyl-2-amino-6-sulfonatonaphthanlene; 4-acetamido-4-isothiocyanatostilbene-2,2′-disulfonic acid; pyrene-3-sulfonic acid; 2-toluidinonapththalene-6-sulfonate; N-phenyl, N-methyl 2-aminonaphthalene-6-sulfonate; ethidium bromide; stebrine; auroniine-0,2-(9′-anthroyl)palmitate; dansyl phosphatidylethanolamin; N,N′-dioctadecyl oxacarbocycanine; N,N′-dihexyl oxacarbocyanine; merocyanine, 4-(3′-pyrenyl)butryate; d-3-aminodesoxy-equilenin; 12-(9′-anthroyl)stearate; 2-methylanthracene; 9-vinylanthracene; 2,2′-(vinylene-p-phenylene)bisbenzoxazole; p-bis[2-(4-methyl-5-phenyl oxazolyl)]benzene; 6-dimethylamino-1,2-benzophenzin; retinol; bis(3′-aminopyridinium)-1,10-decandiyl diiodide; sulfonaphthylhydrazone of hellibrienin; chlorotetracycline; N-(7-dimethylamino-4-methyl-2-oxo-3-chromenyl)maleimide; N-[p-(2-benzimidazolyl)phenyl]maleimide; N-(4-fluoranthyl)maleimide; bis(homovanillic acid); resazarin; 4-chloro-7-nitro-2,1,3-benzooxadizole; merocyanine 540; resorufin; rose bengal and 2,4-diphenyl-3(2H)-furanone. Preferably, the fluorescent detectable moiety is a fluorescein or rhodamine dye.

Another preferred detectable moiety is colloidal gold. The colloidal gold particle is typically 40 to 80 nm in diameter. The colloidal gold may be attached to a labeling compound in a variety of ways. In one embodiment, the linker moiety of the nucleic acid labeling compound terminates in a thiol group (—SH), and the thiol group is directly bound to colloidal gold through a dative bond. See Mirkin et al. Nature 1996, 382, 607-609. In another embodiment, it is attached indirectly, for instance through the interaction between colloidal gold conjugates of antibiotin and a biotinylated labeling compound. The detection of the gold labeled compound may be enhanced through the use of a silver enhancement method. See Danscher et al. J. Histotech 1993, 16, 201-207.

The term “effective amount” as used herein refers to an amount sufficient to induce a desired result.

The term “fragmentation” refers to the breaking of nucleic acid molecules into smaller nucleic acid fragments. In certain embodiments, the size of the fragments generated during fragmentation can be controlled such that the size of fragments is distributed about a certain predetermined nucleic acid length.

The term “genome” as used herein is all the genetic material in the chromosomes of an organism. DNA derived from the genetic material in the chromosomes of a particular organism is genomic DNA. A genomic library is a collection of clones made from a set of randomly generated overlapping DNA fragments representing the entire genome of an organism.

The term “hybridization” as used herein refers to the process in which two single-stranded polynucleotides bind non-covalently to form a stable double-helix polynucleotide; triple-stranded hybridization is also theoretically possible. The resulting (usually) double-stranded polynucleotide is a “hybrid.” The proportion of the population of polynucleotides that forms stable hybrids is referred to herein as the “degree of hybridization.” Hybridizations are usually performed under stringent conditions, for example, at a salt concentration of no more than 1 M and a temperature of at least 25° C. For example, conditions of 5×SSPE (750 mM NaCl, 50 mM NaPhosphate, 5 mM EDTA, pH 7.4) and a temperature of 25-30° C. are suitable for allele-specific probe hybridizations. For stringent conditions, see, for example, Sambrook, Fritsche and Maniatis. “Molecular Cloning A laboratory Manual” 2^{nd} Ed. Cold Spring Harbor Press (1989) which is hereby incorporated by reference in its entirety for all purposes above.

The term “hybridization conditions” as used herein will typically include salt concentrations of less than about 1M, more usually less than about 500 mM and preferably less than about 200 mM. Hybridization temperatures can be as low as 5° C., but are typically greater than 22° C., more typically greater than about 30° C., and preferably in excess of about 37° C. Longer fragments may require higher hybridization temperatures for specific hybridization. As other factors may affect the stringency of hybridization, including base composition and length of the complementary strands, presence of organic solvents and extent of base mismatching, the combination of parameters is more important than the absolute measure of any one alone.

The term “hybridization probes” as used herein are oligonucleotides capable of binding in a base-specific manner to a complementary strand of nucleic acid. Such probes include peptide nucleic acids, as described in Nielsen et al., Science 254, 1497-1500 (1991), and other nucleic acid analogs and nucleic acid mimetics.

The term “hybridizing specifically to” as used herein refers to the binding, duplexing, or hybridizing of a molecule only to a particular nucleotide sequence or sequences under stringent conditions when that sequence is present in a complex mixture (for example, total cellular) DNA or RNA.

The term “isolated nucleic acid” as used herein mean an object species invention that is the predominant species present (i.e., on a molar basis it is more abundant than any other individual species in the composition). Preferably, an isolated nucleic acid comprises at least about 50, 80 or 90% (on a molar basis) of all macromolecular species present. Most preferably, the object species is purified to essential homogeneity (contaminant species cannot be detected in the composition by conventional detection methods).

The term “linker group” (L) as used in connection with the present invention means to provide a linking function, which either alone or in conjunction with appropriate connecting groups, provide appropriate spacing of the Q group from the primary amine (Q-L-NH₂) at such a length and in such a configuration as to allow appropriate reaction with the abasic DNA.

The term “monomer” as used herein refers to any member of the set of molecules that can be joined together to form an oligomer or polymer. The set of monomers useful in the present invention includes, but is not restricted to, for the example of (poly)peptide synthesis, the set of L-amino acids, D-amino acids, or synthetic amino acids. As used herein, “monomer” refers to any member of a basis set for synthesis of an oligomer. For example, dimers of L-amino acids form a basis set of 400 “monomers” for synthesis of polypeptides. Different basis sets of monomers may be used at successive steps in the synthesis of a polymer. The term “monomer” also refers to a chemical subunit that can be combined with a different chemical subunit to form a compound larger than either subunit alone.

The term “mRNA,” sometimes referred to “mRNA transcripts” as used herein, includes, but is not limited to pre-mRNA transcript(s), transcript processing intermediates, mature mRNA(s) ready for translation and transcripts of the gene or genes, or nucleic acids derived from the mRNA transcript(s). Transcript processing may include splicing, editing and degradation. As used herein, a nucleic acid derived from a mRNA transcript refers to a nucleic acid for whose synthesis the mRNA transcript or a subsequence thereof has ultimately served as a template. Thus, a cDNA reverse transcribed from a mRNA, an RNA transcribed from that cDNA, a DNA amplified from the cDNA, an RNA transcribed from the amplified DNA, etc., are all derived from the mRNA transcript and detection of such derived products is indicative of the presence and/or abundance of the original transcript in a sample. Thus, mRNA derived samples include, but are not limited to, mRNA transcripts of a gene or genes, cDNA reverse transcribed from the mRNA, cRNA transcribed from the cDNA, DNA amplified from the genes, RNA transcribed from amplified DNA, and the like.

The term “nucleic acid library,” sometimes referred to as a “array” as used herein refers to a synthetically or biosynthetically prepared collection of nucleic acids. Arrays may be used, inter alia, to screen for the presence or absence of a nucleic acid in a sample. Arrays of nucleic acids are available in a wide variety of different formats (for example, libraries of cDNAs or libraries of oligos tethered to resin beads, silica chips, or other solid supports). Additionally, the term “array” is meant to include those libraries of nucleic acids which can be prepared by spotting nucleic acids of essentially any length (for example, from 1 to about 1000 nucleotide monomers in length) onto a substrate. The term “nucleic acid” as used herein refers to a polymeric form of nucleotides of any length, either ribonucleotides, deoxyribonucleotides or peptide nucleic acids (PNAs), that comprise purine and pyrimidine bases, or other natural, chemically or biochemically modified, non-natural, or derivatized nucleotide bases. The backbone of the polynucleotide can comprise sugars and phosphate groups, as may typically be found in RNA or DNA, or modified or substituted sugar or phosphate groups. A polynucleotide may comprise modified nucleotides, such as methylated nucleotides and nucleotide analogs. The sequence of nucleotides may be interrupted by non-nucleotide components for example by nucleotide analogs that undergo non-traditional hybridization. Thus the terms nucleoside, nucleotide, deoxynucleoside and deoxynucleotide generally include analogs such as those described herein. These analogs are those molecules having some structural features in common with a naturally occurring nucleoside or nucleotide such that when incorporated into a nucleic acid or oligonucleoside sequence, they allow hybridization with a naturally occurring nucleic acid sequence in solution. Typically, these analogs are derived from naturally occurring nucleosides and nucleotides by replacing and/or modifying the base, the ribose or the phosphodiester moiety. The changes can be tailor made to stabilize or destabilize hybrid formation or enhance the specificity of hybridization with a complementary nucleic acid sequence as desired.

The term “nucleic acids” as used herein may include any polymer or oligomer of pyrimidine and purine bases, preferably cytosine, thymine, and uracil, and adenine and guanine, respectively. See Albert L. Lehninger, PRINCIPLES OF BIOCHEMISTRY, at 793-800 (Worth Pub. 1982). Indeed, the present invention contemplates any deoxyribonucleotide, ribonucleotide or peptide nucleic acid component, and any chemical variants thereof, such as methylated, hydroxymethylated or glucosylated forms of these bases, and the like. The polymers or oligomers may be heterogeneous or homogeneous in composition, and may be isolated from naturally-occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states.

The term “oligonucleotide” or sometimes refer by “polynucleotide” as used herein refers to a nucleic acid ranging from at least 2, preferable at least 8, and more preferably at least 20 nucleotides in length or a compound that specifically hybridizes to a polynucleotide. Polynucleotides of the present invention include sequences of deoxyribonucleic acid (DNA) or ribonucleic acid (RNA) which may be isolated from natural sources, recombinantly produced or artificially synthesized and mimetics thereof. A further example of a polynucleotide of the present invention may be peptide nucleic acid (PNA). The invention also encompasses situations in which there is a nontraditional base pairing such as Hoogsteen base pairing which has been identified in certain tRNA molecules and postulated to exist in a triple helix. “Polynucleotide” and “oligonucleotide” are used interchangeably in this application.

The term “photocleavable nucleotide derivative” as used herein with respect to an aspect of the present invention means a 2′-deoxy-nucleotide triphosphate bearing a photocleavable group where said derivative may be incorporated into a growing DNA strand by either DNA polymerase or reverse transcriptase and where upon after photoactivation with electromagnetic radiation of an appropriate wavelength, abasic sites are created in DNA or cDNA.

The term “polymorphism” as used herein refers to the occurrence of two or more genetically determined alternative sequences or alleles in a population. A polymorphic marker or site is the locus at which divergence occurs. Preferred markers have at least two alleles, each occurring at frequency of greater than 1%, and more preferably greater than 10% or 20% of a selected population. A polymorphism may comprise one or more base changes, an insertion, a repeat, or a deletion. A polymorphic locus may be as small as one base pair. Polymorphic markers include restriction fragment length polymorphisms, variable number of tandem repeats (VNTR's), hypervariable regions, minisatellites, dinucleotide repeats, trinucleotide repeats, tetranucleotide repeats, simple sequence repeats, and insertion elements such as Alu. The first identified allelic form is arbitrarily designated as the reference form and other allelic forms are designated as alternative or variant alleles. The allelic form occurring most frequently in a selected population is sometimes referred to as the wildtype form. Diploid organisms may be homozygous or heterozygous for allelic forms. A diallelic polymorphism has two forms. A triallelic polymorphism has three forms. Single nucleotide polymorphisms (SNPs) are included in polymorphisms.

The term “primer” as used herein refers to a single-stranded oligonucleotide capable of acting as a point of initiation for template-directed DNA synthesis under suitable conditions for example, buffer and temperature, in the presence of four different nucleoside triphosphates and an agent for polymerization, such as, for example, DNA or RNA polymerase or reverse transcriptase. The length of the primer, in any given case, depends on, for example, the intended use of the primer, and generally ranges from 15 to 30 nucleotides. Short primer molecules generally require cooler temperatures to form sufficiently stable hybrid complexes with the template. A primer need not reflect the exact sequence of the template but must be sufficiently complementary to hybridize with such template. The primer site is the area of the template to which a primer hybridizes. The primer pair is a set of primers including a 5′ upstream primer that hybridizes with the 5′ end of the sequence to be amplified and a 3′ downstream primer that hybridizes with the complement of the 3′ end of the sequence to be amplified.

The term “probe” as used herein refers to a surface-immobilized molecule that can be recognized by a particular target. See U.S. Pat. No. 6,582,908 for an example of arrays having all possible combinations of probes with 10, 12, and more bases. Examples of probes that can be investigated by this invention include, but are not restricted to, agonists and antagonists for cell membrane receptors, toxins and venoms, viral epitopes, hormones (for example, opioid peptides, steroids, etc.), hormone receptors, peptides, enzymes, enzyme substrates, cofactors, drugs, lectins, sugars, oligonucleotides, nucleic acids, oligosaccharides, proteins, and monoclonal antibodies.

The term “receptor” as used herein refers to a molecule that has an affinity for a given ligand. Receptors may be naturally-occurring or manmade molecules. Also, they can be employed in their unaltered state or as aggregates with other species. Receptors may be attached, covalently or noncovalently, to a binding member, either directly or via a specific binding substance. Examples of receptors which can be employed by this invention include, but are not restricted to, antibodies, cell membrane receptors, monoclonal antibodies and antisera reactive with specific antigenic determinants (such as on viruses, cells or other materials), drugs, polynucleotides, nucleic acids, peptides, cofactors, lectins, sugars, polysaccharides, cells, cellular membranes, and organelles. Receptors are sometimes referred to in the art as anti-ligands. As the term receptors is used herein, no difference in meaning is intended. A “Ligand Receptor Pair” is formed when two macromolecules have combined through molecular recognition to form a complex. Other examples of receptors which can be investigated by this invention include but are not restricted to those molecules shown in U.S. Pat. No. 5,143,854, which is hereby incorporated by reference in its entirety.

The term “solid support”, “support”, and “substrate” as used herein are used interchangeably and refer to a material or group of materials having a rigid or semi-rigid surface or surfaces. In many embodiments, at least one surface of the solid support will be substantially flat, although in some embodiments it may be desirable to physically separate synthesis regions for different compounds with, for example, wells, raised regions, pins, etched trenches, or the like. According to other embodiments, the solid support(s) will take the form of beads, resins, gels, microspheres, or other geometric configurations. See U.S. Pat. No. 5,744,305 for exemplary substrates.

The term “target” as used herein refers to a molecule that has an affinity for a given probe. Targets may be naturally-occurring or man-made molecules. Also, they can be employed in their unaltered state or as aggregates with other species. Targets may be attached, covalently or noncovalently, to a binding member, either directly or via a specific binding substance. Examples of targets which can be employed by this invention include, but are not restricted to, antibodies, cell membrane receptors, monoclonal antibodies and antisera reactive with specific antigenic determinants (such as on viruses, cells or other materials), drugs, oligonucleotides, nucleic acids, peptides, cofactors, lectins, sugars, polysaccharides, cells, cellular membranes, and organelles. Targets are sometimes referred to in the art as anti-probes. As the term targets is used herein, no difference in meaning is intended. A “Probe Target Pair” is formed when two macromolecules have combined through molecular recognition to form a complex.

### C. PHOTOCHEMICAL GENERATION OF ABASIC SITES IN NUCLEIC ACID POLYMERS: RELATED MATERIALS AND METHODS

In one aspect of the invention, methods and compositions are provided for fragmenting a nucleic acid target such as DNA and RNA. In a preferred embodiment, RNA transcripts samples are used as template for a reverse transcription reaction to synthesize cDNAs. The cDNAs may be fragmented and hybridized with a microarray or alternatively, the cDNAs may be used as templates for cDNA synthesis. Methods for synthesizing cDNA are well known in the art. Sample preparation for Whole Transcript Assays are described, for example, in U.S. patent application Ser. No. 10/917,643 which is incorporated herein by reference. Both single-stranded and double-stranded DNA targets may be fragmented. The methods of the invention are particularly suitable for use with arrays that interrogate a large portion of the transcripts, such as tiling arrays, all exon arrays, and alternative splicing arrays.

One of skill in the art would appreciate that the methods and compositions are useful for fragmenting nucleic acids in many applications in addition to assays that measures RNA transcripts. For example, the methods and compositions are also useful for genotyping assays such as the Whole Genome Sampling Assays (WGSA, Affymetrix, Santa Clara) for use with commercially available 10 K or 100 K SNP genotyping arrays.

While the methods of the invention has broad applications and are not limited to any particular detection methods, they are particularly suitable for detecting a large number of, such as more than 1000, 5000, 10,000, 50,000 different transcript features.

Fragmentation of nucleic acids comprises breaking nucleic acid molecules into smaller fragments. Fragmentation of nucleic acid may be desirable to optimize the size of nucleic acid molecules for certain reactions and destroy their three dimensional structure. For example, fragmented nucleic acids may be used for more efficient hybridization of target DNA to nucleic acid probes than non-fragmented DNA. According to a preferred embodiment, before hybridization to a microarray, target nucleic acid should be fragmented to sizes ranging from 50 to 200 bases long to improve target specificity and sensitivity. In a more preferred embodiment, the average size of such fragments, one must consider the components of the assay cocktail in partial fragments obtained is at least 10, 20, 30, 40, 50, 60, 70, 80, 100 or 200 nucleotides. To obtain fragments of such size, molar ratios of cold to hot nucleotides in the reaction mixture must be considered as well as the affinity constant, Kₘ, of the enzyme at issue for the analogs at question and to the substrate. The greater the ratio of hot nucleotide to cold, the greater the level of incorporation that may be expected. The greater the ratio of incorporation of photoactive nucleotides, the smaller the size of resulting fragments.

However, there are practical limitations to simply increasing the molar ration of hot nucleotides to cold ones. For example, some analogs, including photocleavable analogs may act as enzyme inhibitors. Thus, high levels of photonucleotides may simply inhibit the reverse transcriptase While many theoretical predictions can be made regarding RNA transcription, persons of skill in the art will generally attempt to determine empirically the best conditions to use for a particular sample, hot nucleotide, mRNA or gene of interest and nucleotide array to be used. Preferably, using the empirical approach, all factors are held steady save one which is varied until optimal results are obtained. Then, the next factor can be examined and varied. Thus, for example, increasing the molarity of the photocleavable nucleotide derivative, while holding the concentrations or molarities of the cold nucleotides constant and plotting the data on incorporation versus molarity of the photocleavable group may yield valuable information as to the appropriate amount of photocleavable group to use for obtaining the desired fragment length.

Alternatively, in accordance with an aspect of the present invention, still more information maybe gleaned by varying the concentrations of the cold nucleotides as well as the amount of enzyme used. Other factors would occur to those of ordinary skill in the art. For example the temperature of the reaction condition could be important. In this regard, nucleotide derivatives do not undergo traditional Watson-Crick base pairing with their counterparts. This is in turn could lead to decreased activity. Heat might alleviate this problem.

Another factor that would occur to the person of skill in the art is the time of the reaction. Again, nucleotide derivatives do not undergo perfect Watson-Crick base pairing. Incubating the reactants might allow for greater incorporation.

It should be noted that there are a number of assays determine incorporation by the nucleotide derivative. For example, pure chemical assays can be performed simply to determine incorporation of the nucleotide derivative into a cDNA. On the other end of the spectrum, biological experiments can be conducted where it is determined whether the incorporated and labeled cRNA can be hybridized to a nucleic acid array to generate a hybridization pattern. The hybridization pattern can then be examined to determine the pattern of expression or a genotype.

In accordance with an aspect of the present invention discussed in numerous references including those incorporated by reference, labeling may be performed before or at the same time as fragmentation using. Labeling methods are well known in the art and are what happened to rest of sentence?

In one preferred embodiment of the present invention, the products of the fragmentation methods are substrates for 3′ end labeling with Affymetrix biotinylated DNA Labeling Reagent (DLR—Affymetrix, Santa Clara, Calif., USA), described above, using the enzyme terminal deoxynucleotidyl transferase (TdT) (aka Terminal Transferase). Labeled dNTPs can be incorporated this way onto the 3′-OH end of DNA in a template independent reaction. See also, U.S. Patent Application Nos. 60/545,417, 60/542,933, 10/452,519 and 10/617,992.

One of skill in the art will appreciate that in order to measure the transcription level (and thereby the expression level) of a gene or genes, it is desirable to provide a nucleic acid sample comprising mRNA transcript(s) of the gene or genes, or nucleic acids derived from the mRNA transcript(s). As used herein, a nucleic acid derived from a mRNA transcript refers to a nucleic acid which is homologous to the mRNA or to an anti-sense strand homologous to the mRNA.

Thus, a cDNA reverse transcribed from a mRNA, a cRNA transcribed from that cDNA, a DNA reverse transcribed from the cRNA, etc., are all derived from the mRNA transcript and detection of such derived products is indicative of the absence, presence and/or abundance of the original transcript in a sample. Thus, suitable samples include, but are not limited to, mRNA transcripts, cDNA reverse transcribed from the mRNA, cRNA transcribed from the cDNA, and DNA reverse transcribed from cRNA.

The above procedures provide some advantages in detecting RNA. See, e.g., U.S. Ser. No. 10/917,643. First, the original mRNA, which might be exceedingly rare, is amplified to provide at least a moderate copy number of the nucleic acid of interest. The mRNA is hybridized to a primer (random or oligo poly dT coupled to a bacterial RNA promoter such as T7, for example without limitation). After second strand formation, the promoter can be used to amplify the original mRNA by having the promoter generate a multitude of cRNA copies of the original sequence. These techniques are familiar to those of skill in the art.

In accordance with an aspect of the present invention, the cRNA is then converted back to DNA by a second round of reverse transcription. DNA is generally more stable than RNA and has a variety of labeling pathways. To convert cRNA back to DNA, the cRNA is hybridized with random primers. The primers are then extended with reverse transcriptase. Reverse transcriptases are capable of incorporating a number of different type of DNA analogs. Moreover, 3′-OH groups of DNA can be labeled with biotin by Trerminal Transferase.

In a particularly preferred embodiment, where it is desired to quantify the transcription level (and thereby expression) of a one or more genes in a sample, the nucleic acid sample is one in which the concentration of the mRNA transcript(s) of the gene or genes, or the concentration of the nucleic acids derived from the mRNA transcript(s), is proportional to the transcription level (and therefore expression level) of that gene. Similarly, it is preferred that the hybridization signal intensity be proportional to the amount of hybridized nucleic acid. While it is preferred that the proportionality be relatively strict (e.g., a doubling in transcription rate results in a doubling in mRNA transcript in the sample nucleic acid pool and a doubling in hybridization signal), one of skill will appreciate that the proportionality can be more relaxed and even non-linear. Thus, for example, an assay where a 5 fold difference in concentration of the target mRNA results in a 3 to 6 fold difference in hybridization intensity is sufficient for most purposes. Where more precise quantification is required appropriate controls can be run to correct for variations introduced in sample preparation and hybridization as described herein. In addition, serial dilutions of “standard” target mRNAs can be used to prepare calibration curves according to methods well known to those of skill in the art. Of course, where simple detection of the presence or absence of a transcript is desired, no elaborate control or calibration is required.

In the simplest embodiment, such a nucleic acid sample is the total mRNA isolated from a biological sample. The term “biological sample”, as used herein, refers to a sample obtained from an organism or from components (e.g., cells) of an organism. The sample may be of any biological tissue or fluid. Frequently the sample will be a “clinical sample” which is a sample derived from a patient. Such samples include, but are not limited to, sputum, blood, blood cells (e.g., white cells), tissue or fine needle biopsy samples, urine, peritoneal fluid, and pleural fluid, or cells there from. Biological samples may also include sections of tissues such as frozen sections taken for histological purposes.

The nucleic acid (either genomic DNA or mRNA) may be isolated from the sample according to any of a number of methods well known to those of skill in the art. One of skill will appreciate that where alterations in the copy number of a gene are to be detected genomic DNA is preferably isolated. Conversely, where expression levels of a gene or genes are to be detected, preferably RNA (mRNA) is isolated.

Methods of isolating total mRNA are well known to those of skill in the art. For example, methods of isolation and purification of nucleic acids are described in detail in Chapter 3 of Laboratory Techniques in Biochemistry and Molecular Biology: Hybridization With Nucleic Acid Probes, Part I. Theory and Nucleic Acid Preparation, P. Tijssen, ed. Elsevier, N.Y. (1993) and Chapter 3 of Laboratory Techniques in Biochemistry and Molecular Biology: Hybridization with Nucleic Acid Probes, Part I. Theory and Nucleic Acid Preparation, P. Tijssen, ed. Elsevier, N.Y. (1993)).

According to an aspect of the present invention, total nucleic acid is isolated from a given sample using, for example, an acid guanidinium-phenol-chloroform extraction method and polyA⁺ mRNA is isolated by oligo dT column chromatography or by using (dT)n magnetic beads (see, e.g., Sambrook et al., Molecular Cloning: A Laboratory Manual (2nd ed.), Vols. 1-3, Cold Spring Harbor Laboratory, (1989), or Current Protocols in Molecular Biology, F. Ausubel et al., ed. Greene Publishing and Wiley-Interscience, New York (1987)).

Frequently, it is desirable to amplify the nucleic acid sample prior to hybridization. One of skill in the art will appreciate that whatever amplification method is used, if a quantitative result is desired, care must be taken to use a method that maintains or controls for the relative frequencies of the amplified nucleic acids.

Methods of “quantitative” amplification are well known to those of skill in the art. For example, quantitative PCR involves simultaneously co-amplifying a known quantity of a control sequence using the same primers. This provides an internal standard that may be used to calibrate the PCR reaction. The high density array may then include probes specific to the internal standard for quantification of the amplified nucleic acid.

One preferred internal standard is a synthetic AW106 cRNA. The AW106 cRNA is combined with RNA isolated from the sample according to standard techniques known to those of skill in the art. The RNA is then reverse transcribed using a reverse transcriptase to provide copy DNA. The cDNA sequences are then amplified (e.g., by PCR) using labeled primers. The amplification products are separated, typically by electrophoresis, and the amount of radioactivity (proportional to the amount of amplified product) is determined. The amount of mRNA in the sample is then calculated by comparison with the signal produced by the known AW106 RNA standard. Detailed protocols for quantitative PCR are provided in PCR Protocols, A Guide to Methods and Applications, Innis et al., Academic Press, Inc. N.Y., (1990).

Other suitable amplification methods include, but are not limited to polymerase chain reaction (PCR) (Innis, et al., PCR Protocols. A guide to Methods and Application. Academic Press, Inc. San Diego, (1990)), ligase chain reaction (LCR) (see Wu and Wallace, Genomics, 4: 560 (1989), Landegren, et al., Science, 241: 1077 (1988) and Barringer, et al., Gene, 89: 117 (1990), transcription amplification (Kwoh, et al., Proc. Natl. Acad. Sci. USA, 86: 1173 (1989)), and self-sustained sequence replication (Guatelli, et al., Proc. Nat. Acad. Sci. USA, 87: 1874 (1990)).

Methods of in vitro polymerization are well known to those of skill in the art (see, e.g., Sambrook, supra.) and this particular method is described in detail by Van Gelder, et al., Proc. Natl. Acad. Sci. USA, 87: 1663-1667 (1990) who demonstrate that in vitro amplification according to this method preserves the relative frequencies of the various RNA transcripts. Moreover, Eberwine et al. Proc. Natl. Acad. Sci. USA, 89: 3010-3014 provide a protocol that uses two rounds of amplification via in vitro transcription to achieve greater than 10⁶ fold amplification of the original starting material thereby permitting expression monitoring even where biological samples are limited.

It will be appreciated by one of skill in the art that the direct transcription method described above provides an antisense (aRNA) pool. Where antisense RNA is used as the target nucleic acid, the oligonucleotide probes provided in the array are chosen to be complementary to subsequences of the antisense nucleic acids. Conversely, where the target nucleic acid pool is a pool of sense nucleic acids, the oligonucleotide probes are selected to be complementary to subsequences of the sense nucleic acids. Finally, where the nucleic acid pool is double stranded, the probes may be of either sense as the target nucleic acids include both sense and antisense strands.

The protocols cited above include methods of generating pools of either sense or antisense nucleic acids. Indeed, one approach can be used to generate either sense or antisense nucleic acids as desired. For example, cDNA can be directionally cloned into a vector (e.g., Stratagene's p Bluscript II KS (+) phagemid) such that it is flanked by the T3 and T7 promoters. In vitro transcription with the T3 polymerase will produce RNA of one sense (the sense depending on the orientation of the insert), while in vitro transcription with the T7 polymerase will produce RNA having the opposite sense. Other suitable cloning systems include phage lamda vectors designed for Cre-loxP plasmid subcloning (see e.g., Palazzolo et al., Gene, 88: 25-36 (1990)).

In a particularly preferred embodiment, a high activity RNA polymerase (e.g. about 2500 units/.mu.L for T7, available from Epicentre Technologies) is used.

Nucleic Acid Labeling

Reverse transcriptases, DNA polymerases, RNA polymerases and their mutants can incorporate certain modified dNTPs or rNTPs to some extent (Kukhanova, M.; et al., Biochemica et Biophysica Acta, 1986, 868, 136-144; Sousa, R.; Padilla, R., et al. Nucleic Acids Research, 2002, Vol. 30, No. 24 e138; Khorana, H. G.; et al., J. Biol. Chem. 1972, 247, 6140-6148; Goeff, S. P.; et al., Proc. Natl. Aca. Sci. USA 1997, 94, 407-41; Suzuki, M.; et al., Mutation Research 2001, 485, 197-207), each of which are incorporated herein by reference for all purposes. Reverse transcriptases, DNA polymerases and their mutants can incorporate dye-dNTP's as well. Holliger, P.; et al., Nature Biotechnology, 2004, 22, 755-759 and references cited therein. Each of the above references is incorporated herein by reference for all purposes.

However, the ability of DNA polymerases to incorporate base analogs is limited. For example, 5-nitroindol-2′-deoxyribose-5′-triphosphate (1) is incorporated by polymerases, but acts as a chain terminator. See Loakes, D., Nucleic. Acids Research, 2001, 29, 2437-2447; and Smith, C. L.; Nucl. 1998, 17, 541-554.

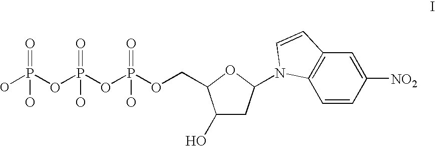

Photochemically cleavable nucleotides are known in the art. For example, 3-nitro-3-deaza-2′-deoxyadenosine inserted as 2 by solid-phase phosphoramidite chemistry into single or double-stranded DNA has been shown to undergo site-specific photochemical cleavage resulting in 3′ and 5′-phosphorylated fragments. See Kotera, M; et al., J. Amer. Chem. Soc. 2004, 126, 9532-9533; Kotera, M.; et al., J. Amer. Chem. Soc. 1998, 120, 11810-11811; and Kotera, M.; et al., J. Amer. Chem. Soc. 2002, 124, 9129-9135.

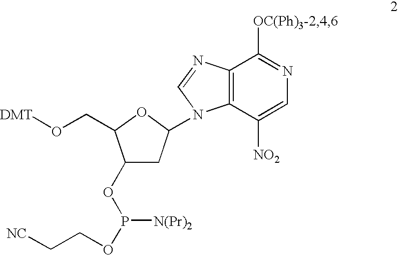

In accordance with an aspect of the present invention, deoxyribonucleoside triphosphates bearing a nitro group such as by way of example and not limiting 3 may be used as substrates for reverse transcriptase and DNA polymerase or their mutants in which the analog is internally incorporated into DNA: 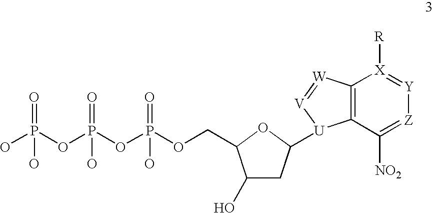
where U, V, W, X, Y, Z are C or N or any combination thereof, and R is either H, OH or NH2 if X═C, and R is a pair of non-bonded electrons if X═N.

The modified DNA can then undergo photolysis at 365 nm resulting in generation of a 2′-deoxyribonolactone lesion (abasic site) which can be cleaved enzymatically or chemically for subsequent labeling, or labeled directly with a molecule containing a primary amino group. See, e.g., Kotera, M; et al., J. Amer. Chem. Soc. 2004, 126, 9532-9533; Kotera, M.; et al., J. Amer. Chem. Soc. 1998, 120, 11810-11811; and Kotera, M.; et al., J. Amer. Chem. Soc. 2002, 124, 9129-9135.

In accordance with an aspect of the present invention, this deoxyribonolactone can be excised with class II endonucleases, such as Endo IV, for TdT labeling of the 3′-OH fragment. In yet another embodiment of the present invention, the deoxyribonolactone can be excised with base (Kotera, M.; et al., J. Amer. Chem. Soc. 2002, 124, 9129-9135) followed by phosphatase treatment for TdT end-labeling. In still a further embodiment of the present invention, the deoxyribonolactone can be labeled directly with R-L-NH₂ molecules, where R is a reporter group and L is a linker (U.S. patent application Ser. No. 10/951,983). Most preferably, 3-nitro-3-deaza-2′-deoxyadenosine triphosphate 4 is used as a substrate for any DNA polymerase or any reverse transcriptase or their mutant forms:

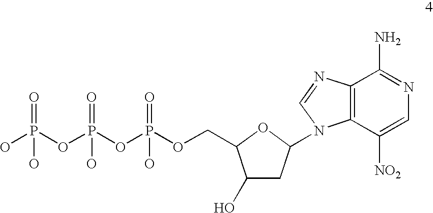

This approach should have the same benefit as the UDG/dUTP cleavage method in that the fragmentation is robust (fragmentation to a reaction end-point) (U.S. patent application Ser. No. 10/951,983). Scheme I shows incorporation of 3-nitro-3-deaza-2′-deoxyadenosine triphosphate 4 into a growing strand by a DNA polymerase or reverse transcriptase followed by photolytic cleavage of the base to leave an abasic lesion (Kotera, M; et al., J. Amer. Chem. Soc. 2004, 126, 9532-9533):

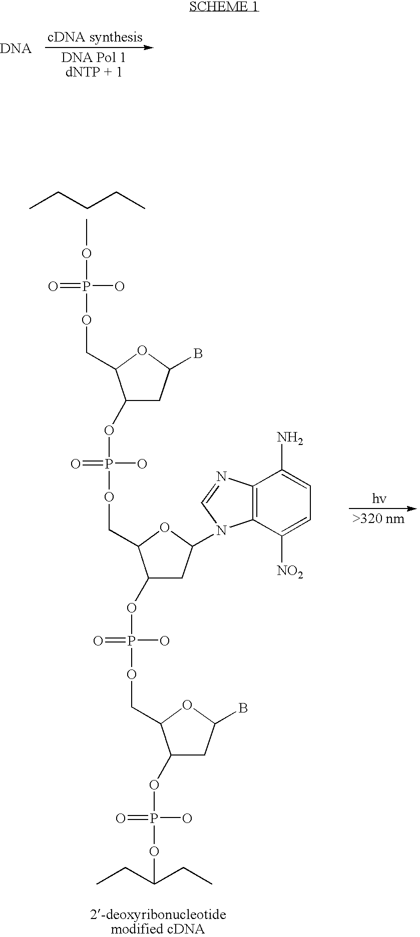

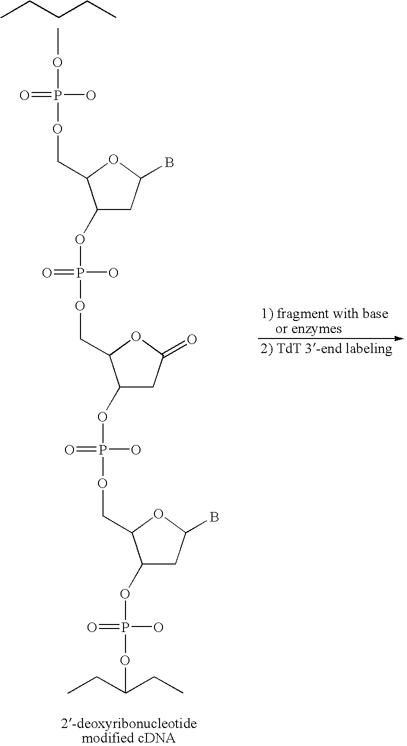

In accordance with an aspect of the present invention, the 2′-deoxyribolactone modified cDNA can be labeled in at least three separate ways. First, the lactone chain may be treated with Endonuclease IV, followed by labeling with terminal transferase. Endonuclease IV from Escherichia coli is a 32 kD metalloprotein that aids in the repair of damaged DNA. The enzyme functions both as an apurinic/apyrimidinic nuclease (Ljungquist, S. (1977) J. Biol. Chem. 252, 2808) and as a 3′ terminal diesterase. See Ljungquist, S. (1977) J. Biol. Chem. 252, 2808; Demple, B. et al., (1986) Proc. Natl. Acad. Sci. USA 83, 7731; Levin, J. D. et al., (1988) J. Biol. Chem. 263, 8066; and Levin, J. D. et al., (1991) J. Biol. Chem. 266, 22893. The latter activity is important in the repair of DNA strand breaks generated by oxidation (e.g., H2O2) and ionic radiation. In such events, the strand breaks terminate with either a 3′ phosphate or a deoxyribose fragment, preventing repair by DNA polymerase I or DNA ligase. Endonuclease IV removes the blocking groups, leaving a free 3′ hydroxyl terminus. Although a metalloenzyme, Endonuclease IV is active in the presence of EDTA provided a suitable substrate is present. In addition, the enzyme does not have detectable associated exonuclease or DNA N-glycosylase activities.

Following treatment with Endonuclease IV, a 3′ hydroxyl group is generated. In accordance this aspect of the present invention, terminal transferase is used to add a detectable moiety to the 3′ end. The detectable moiety is preferably biotin. Most preferably, the molecule used to add the 3′ biotin as the following abasic triphosphate: 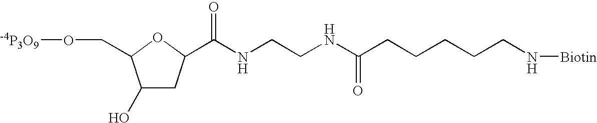
The above molecule (termed herein as DLR) is described in detail in U.S. patent Ser. No. 10/314,012.

In accordance with another aspect of the present invention, the lactone chain may be broken by treatment with base. This, however, leaves a phosphate on the 3′ end of the sugar which is not a substrate for terminal transferase. Hence, this phosphate must first be removed via the appropriate phosphatase. Then, terminal transferase may be used to add DLR and biotinylate the strand.

In yet another embodiment of the present invention, the lactone may directly attacked by treatment with for example a primary amine bearing a detectable moiety such as biotin. Scheme II shows this reaction:

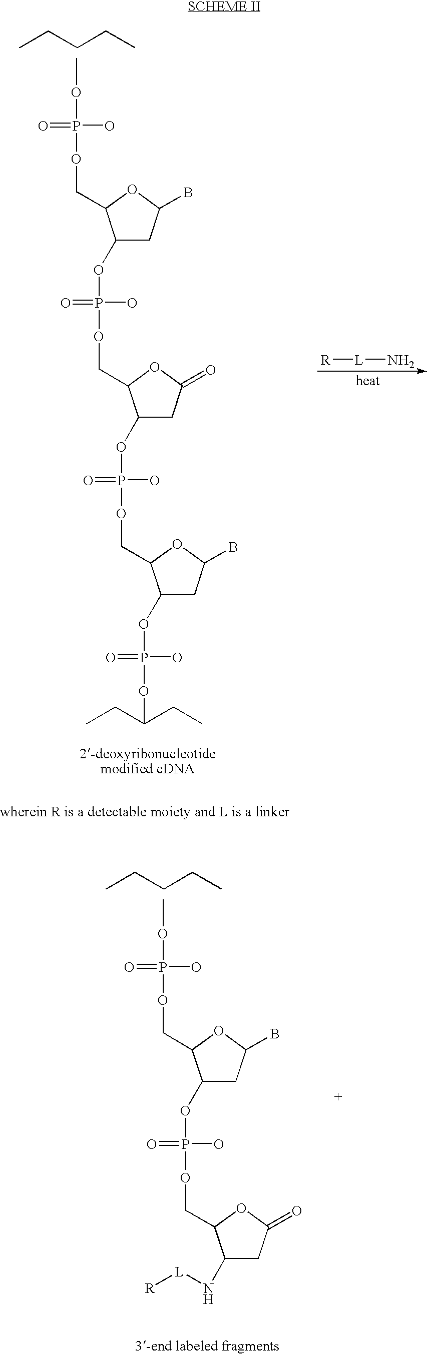

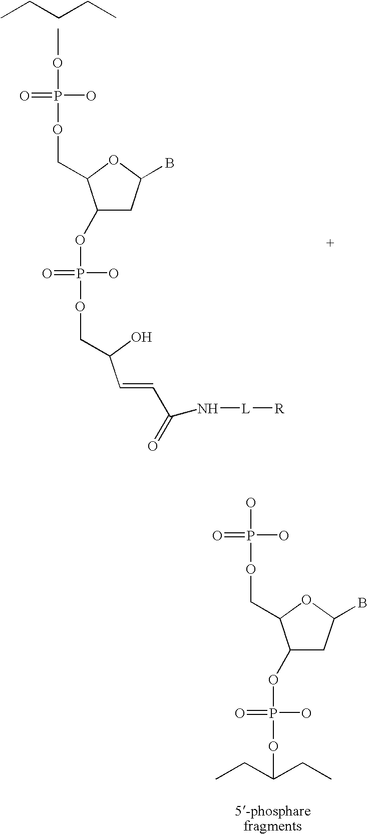

Random oligomer primers for use in the present invention can be custom made, “off the shelf” or “home” made. The primers can be from about 6 to about 15 nucleotides in length. The amount of primer used will affect efficiency and the length of synthesized products. The range of weight ratios of hexamer to initial RNA input should be between about 1:100 and 10:1, preferably about 1:10. Higher ratios tend to yield shorter products. Enzymes which can be used to synthesize second strand cDNA are any known in the art for such purpose. E. coli DNA polymerase I can be used, as well as Klenow fragment. These can optionally be used with DNA ligase which will promote longer fragments a second part comprising a strong promoter sequence. Typically the strong promoter is from a bacteriophage, such as SP6, T7 or T3. Promoters which drive robust in vitro transcription are desirable. Because most populations of MRNA from biological samples do not share any sequence homology other than a poly(da) tract at the 3′ end, the first part of the primer typically comprises a poly(dT) sequence which is generally complementary to most mRNA species. The length of the tract is typically from about 5 to 20 nucleotides, more preferably about 10 to 15 nucleotides. Alternatively, if a subpopulation of RNA is desired, a primer which is complementary to a common sequence feature in the subpopulation can be used. Yet another type of priming employs random oligomers. Such oligomers should yield a full and representative set of cDNA. The orientation of the promoter sequence is important. It is typically at the 5′ end of the primer, so that the 3′ end can successfully anneal and drive reverse transcription. Moreover, the promoter sequence is oriented in such a fashion that it is “opposite” the 3′ end of the MRNA. Thus upon second strand synthesis, the double stranded promoter will be at the 3′ end of the gene, in an orientation favorable for producing reverse strand (negative strand, or antisense) RNA. This orientation is termed “antisense” orientation. Hybrids of first strand cDNA and MRNA can be denatured according to any method known in the art. These include the use of heat and the use of alkali. Heat treatment is the preferred method. Denaturation is desirable until less than 50% of the hybrids remain annealed. More denaturation is desirable, such as until less than 75%, 85% or 95% of the hybrids remain annealed as hybrids.

Quantitation of particular RNA molecules within the population of copy RNA can be done according to any means known in the art. These include but are not limited to Northern blotting and hybridization to nucleic acid arrays. Typically, some sort of hybridization step must be involved to provide the specificity required to measure transcripts individually. Alternatively, the cRNA can be reverse transcribed into cDNA and a specific cDNA species can be amplified to obtain specificity. Copy RNA can be used for any use known in the art, not merely quantitation. It can be used for cloning, and/or expression, or as a probe. Such uses can be applied to determining a diagnosis or prognosis, to determining an etiological basis for disease, for determining a cell type or species source, for identifying infectious organisms in foods, hospitals, ventilation systems, and for testing drugs for their main or side effects. Other applications will be readily apparent to those of skill in the art.

Useful labels in the present invention include biotin for staining with labeled streptavidin conjugate, magnetic beads (e.g., Dynabeads™), fluorescent dyes (e.g., fluorescein, texas red, rhodamine, green fluorescent protein, and the like), radiolabels (e.g., .sup.3 H, .sup.125 I, .sup.35 S, .sup. 14 C, or .sup.32 P), enzymes (e.g., horse radish peroxidase, alkaline phosphatase and others commonly used in an ELISA), and colorimetric labels such as colloidal gold or colored glass or plastic (e.g., polystyrene, polypropylene, latex, etc.) beads. Patents teaching the use of such labels include U.S. Pat. Nos. 3,817,837; 3,850,752; 3,939,350; 3,996,345; 4,277,437; 4,275,149; and 4,366,241.

The label may be added to the target (sample) nucleic acid(s) prior to, or after the hybridization. So called “direct labels” are detectable labels that are directly attached to or incorporated into the target (sample) nucleic acid prior to hybridization. In contrast, so called “indirect labels” are joined to the hybrid duplex after hybridization. Often, the indirect label is attached to a binding moiety that has been attached to the target nucleic acid prior to the hybridization. Thus, for example, the target nucleic acid may be biotinylated before the hybridization. After hybridization, an aviden-conjugated fluorophore will bind the biotin bearing hybrid duplexes providing a label that is easily detected. For a detailed review of methods of labeling nucleic acids and detecting labeled hybridized nucleic acids see Laboratory Techniques in Biochemistry and Molecular Biology, Vol. 24: Hybridization With Nucleic Acid Probes, P. Tijssen, ed. Elsevier, N.Y., (1993)).

A nucleic acid array according to the present invention is any solid support having a plurality of different nucleotide sequences attached thereto or associated therewith. One preferred type of nucleic acid array that is useful in the present invention include those that are commercially available from Affymetrix (Santa Clara, Calif.) under the brand name GeneChip®. Example arrays are shown on the website at affymetrix.com.

GeneChip Analysis.

GeneChip® nucleic acid probe arrays are manufactured using technology that combines photolithographic methods and combinatorial chemistry. In a preferred embodiment, over 280,000 different oligonucleotide probes are synthesized in a 1.28 cm×1.28 cm area on each array. Each probe type is located in a specific area on the probe array called a probe cell. Measuring approximately 24 μm×24 μm, each probe cell contains more than 10⁷ copies of a given oligonucleotide probe.

Probe arrays are manufactured in a series of cycles. A glass substrate is coated with linkers containing photolabile protecting groups. Then, a mask is applied that exposes selected portions of the probe array to ultraviolet light. Illumination removes the photolabile protecting groups enabling selective nucleotide phosphoramidite addition only at the previously exposed sites. Next, a different mask is applied and the cycle of illumination and chemical coupling is performed again. By repeating this cycle, a specific set of oligonucleotide probes is synthesized, with each probe type in a known physical location. The completed probe arrays are packaged into cartridges.

In accordance with an aspect of the present invention, a method is presented for detecting the presence or absence of a mRNA in a nucleic acid sample by hybridization to a nucleic acid array, the method comprising the steps of providing a nucleic acid sample comprising mRNA; hybridizing the mRNA with an oligonucleotide primer comprising an oligonucleotide homologous to said mRNA; providing a 2′-deoxynucleotide triphosphate derivative having an azido group allowing for the chemical attachment of a phosphone derivatized detectable label; reverse transcribing said mRNA with a reverse capable of incorporating the deoxynucleotide derivative with a rate and fidelity substantially similar to that for natural 2′ deoxynucleotide triphosphates to provide reverse transcribed DNA homologous to all or part of said mRNA having azido groups; reacting the azido groups on the DNA with a phosphone derivatized detectable label to provide labeled DNA; and hybridizing the labeled DNA to said nucleic acid array to detect the presence or absence of the mRNA.

Reverse transcription is performed according to an aspect of the present invention according to standard techniques known in the art. The reaction is typically catalyzed by an enzyme from a retrovirus, which is competent to synthesize DNA from an RNA template. According to the present method, the primer used for the first round of reverse transcription has two parts: one part for annealing to the RNA molecules through Watson-Crick base pairing and a second portion comprising a strong promoter sequence. Typically the strong promoter is from a bacteriophage, such as SP6, T7 or T3. Promoters which drive robust in vitro transcription are desirable. Preferably, T7 is used.

Because most populations of mRNA from biological samples do not share any sequence homology other than a poly(dA) tract at the 3′ end, the first part of the primer typically comprises a poly(dT) sequence which is complementary to many mRNA species (addition of a poly A tail to a mRNA is a typical RNA processing event for mature mRNAs that will be translated into protein.) The length of the tract is typically from about 5 to 20 nucleotides, more preferably about 10 to 15 nucleotides. Alternatively, if a subpopulation of RNA is desired, a primer which is complementary to a common sequence feature in the subpopulation can be used.

Yet another technique of mRNA promoting is the use of random primers. This technique is known to those of skill in the art and has the advantage of not being dependent on the presence of poly A sequences. Many RNA's, do not contain poly A+ tracts. Thus, the use of poly dT results in under representation of RNAs in the cell. The orientation of the promoter sequence is important. Typically, the 3′ end of the primer is used to drive reverse transcription. Moreover, the promoter sequence is oriented in such a fashion that it is “opposite” the 3′ end of the mRNA. Thus upon second strand synthesis, the double stranded promoter will be at the 3′ end of the gene, in an orientation favorable for producing reverse strand (negative strand, or antisense) RNA. This orientation is termed “antisense” orientation. Hybrids of first strand cDNA and mRNA can be denatured according to any method known in the art. These include the use of heat and the use of alkali. Heat treatment is the preferred method. Denaturation is desirable until less than 50% of the hybrids remain annealed. More denaturation is desirable, such as until less than 75%, 85% or 95% of the hybrids remain annealed as hybrids.

Transcription of the double stranded cDNA molecules is a linear process which creates large amounts of product from small input amount, without greatly distorting the relative amounts of input. Thus the transcription process while being efficient is “linear” rather than “exponential.” Labeled ribonucleotides can be used during transcription of the double stranded cDNA. These can be radioactively labeled, with such isotopes as ³²P, ³H, and ³⁵S. Alternatively, in accordance with the present invention, cRNA can be concerted back to DNA via hybridization to random primers. Photonucleotide triphosphates can be incorporated into the second round cDNA synthesis in accordance with the present invention as described above. After incorporation of these photonucleotide groups various stratagems can be employed to cleave the DNA into fragments followed by labeling the fragments with a detectable moiety. Preferably, in accordance with the present invention, the detectable moiety is biotin, incorporated from DLR.

The labeled avidin can contain any desirable and convenient detectable label. Quantitation of particular RNA molecules within the population of copy RNA can be done according to any means known in the art. These include but are not limited to Northern blotting and hybridization to nucleic acid arrays. Typically, some sort of hybridization step must be involved to provide the specificity required to measure transcripts individually. Alternatively, the cRNA can be reverse transcribed into cDNA and a specific cDNA species can be amplified to obtain specificity. Copy RNA can be used for any use known in the art, not merely quantitation. It can be used for cloning, and/or expression, or as a probe. Such uses can be applied to determining a diagnosis or prognosis, to determining an etiological basis for disease, for determining a cell type or species source, for identifying infectious organisms in foods, hospitals, ventilation systems, and for testing drugs for their main or side effects. Other applications will be readily apparent to those of skill in the art.

Generally, in accordance with the present invention, the reverse transcriptase should be capable of incorporating the deoxynucleotide derivative, i.e., the photochemical nucleotide derivative, into a growing cDNA strand with a rate and fidelity substantially similar to that for natural 2′ deoxynucleotide triphosphates. However, this is both a flexible and a practical requirement. For example, depending on the mRNA to be detected, the enzyme might work at an order of magnitude lower than the same enzyme with wildtype substrates. However, this level of activity may still be sufficient to fragment and label the DNA strand as required in accordance with an aspect of the present invention. The ultimate requirement is that the enzyme/substrate combination provide a workable labeling system, considering the rate of incorporation and the fidelity of incorporation, i.e. that the template be copied with a relatively small number of errors. In this regard, for example, a G or G analog should be incorporated by the reverse transcriptase when a C is presented on the mRNA template. Also, the rate of the reaction must be maintained so that the assay can be carried out in a reasonable period of time, e.g., a total time of 24-48 hours. However, these are not absolute requirements. Rather, they are guideposts to those of skill in the art in determining appropriate enzyme, substrate combinations.

In accordance with an aspect of the present invention, a method is presented for analyzing a nucleic acid sample containing mRNA, the method having the following steps: providing a nucleic acid sample containing mRNA; synthesizing cDNA in the presence of a photocleavable nucleotide derivative, wherein said photocleavable nucleotide derivative provides abasic DNA upon incorporation into a DNA strand, following exposure to light of an appropriate wavelength; exposing said cDNA to light of a predetermined wavelength to cause photocleavage and formation of a plurality of abasic sites to provide abasic cDNA; cleaving said abasic cDNA with an endonuclease as to generate a plurality of fragments with terminal free 3′ hydroxyl groups; labeling said fragments with biotin using terminal transferase; hybridizing said labeled fragments to a nucleic acid array to provide a hybridization pattern; and analyzing the hybridization pattern.

Preferably, the photocleavable nucleotide derivative is 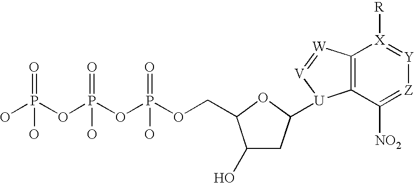
wherein U, V, W, X, Y and Z are C or N or any combination thereof, R is H, OH or NH₂, wherein if R is NH₂, X is C and wherein if X is N, R is a pair of non-bonded electrons. The photocleavable nucleotide derivative preferably has the structure 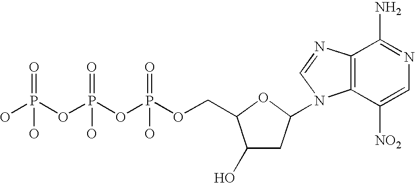
The photocleavable protecting group is preferably cleaved with light having a wavelength of from 320 nm up to approximately 380 nm. More preferably, the light has a wavelength of about 365 nm.

According to the method of one aspect of the instant invention, the endonuclease is endonuclease IV. In another preferred embodiment of the instant invention, the endonuclease is endonuclease ApeI.

According to an aspect of the present invention, the cDNA is cleaved at abasic sites by endonuclease V.

Preferably, the fragments are generated having an average size range selected from the group consisting of 10, 20, 30, 40, 50, 60,70, 80, 100 or 200 nucleotides. In accordance with instantly disclosed methods the cleaving and the labeling steps are preferably carried out simultaneous.

In accordance with the present invention, cDNA is preferably ss-cDNA. In another preferred embodiment of the instant invention, cDNA is preferably ds-cDNA.

In accordance with another preferred embodiment of the present invention, the photocleavable nucleotide is preferably 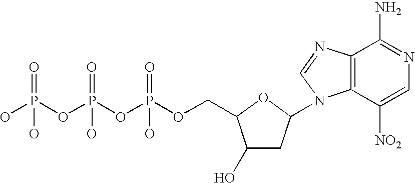
which is preferably incorporated into the ss-cDNA during reverse transcription. In another preferred embodiment of the instant invention, 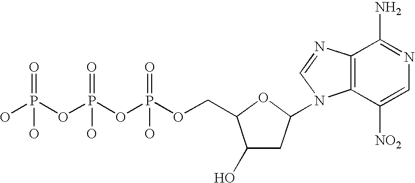
   is incorporated into the ds-cDNA during second strand cDNA synthesis. According to yet another preferred embodiment of the instant invention, 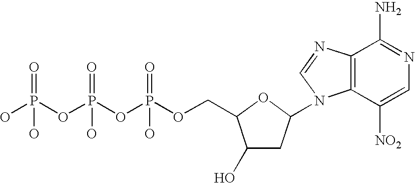
   is incorporated in a single or in both strands of ds-cDNA.

In yet another preferred aspect of the instant invention, a method for analyzing a nucleic acid sample containing RNA is presented, the method has the following steps: providing a nucleic acid sample containing RNA; synthesizing cDNA in the presence of a photocleavable nucleotide derivative, wherein said photocleavable nucleotide derivative provides abasic DNA upon incorporation into a DNA strand, following exposure to light of an appropriate wavelength; exposing the cDNA to light of a predetermined wavelength to cause photocleavage and formation of a plurality of abasic sites to provide abasic cDNA; incubating the abasic DNA in basic conditions to provide DNA fragments having 3′ terminal phosphate groups; dephosphorylating the fragments to provide 3′ terminal OH groups; and labeling the fragments with biotin using terminal transferase; hybridizing the labeled fragments to a nucleic acid array to provide a hybridization pattern; and analyzing the hybridization pattern. According to this aspect of the present invention, a photocleavable nucleotide derivative is 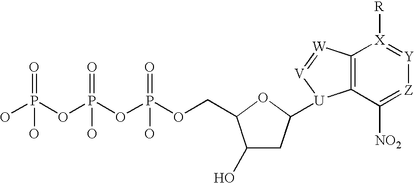
wherein U, V, W, X, Y and Z are C or N or any combination thereof, R is H, OH or NH₂, wherein if R is NH2, X is C and wherein if X is NH2, R is a pair of non-bonded electrons.

Preferably, the photocleavable nucleotide derivative has the structure

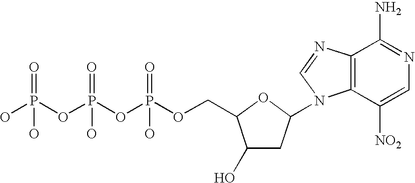

The photocleavable nucleotide derivative above is preferably cleaved by light having a wavelength of from 320 nm up to approximately 380 nm. More preferably, it is cleaved by light with a wavelength of about 365 nm.

Preferably, the nucleic acid sample is mRNA. It is also preferred that the cDNA is ss-cDNA. In another preferred embodiment of the instant invention, the cDNA is ds-cDNA.

In a particularly preferred embodiment of the instant invention, the photocleavable nucleotide has the structure 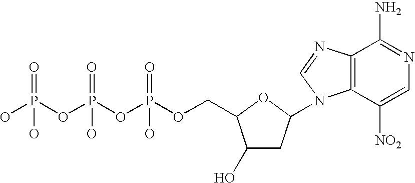
and is incorporated into the ss-cDNA during reverse transcription and into ds-cDNA during second strand cDNA synthesis or into both.

In yet another aspect of the instantly method for analyzing a nucleic acid sample containing RNA, said method having the following steps: providing a nucleic acid sample containing RNA; synthesizing cDNA in the presence of a photocleavable nucleotide derivative, wherein said photocleavable nucleotide derivative provides abasic DNA upon incorporation into a DNA strand, following exposure to light of an appropriate wavelength; exposing the cDNA to light of a predetermined wavelength to cause photocleavage and formation of a plurality of abasic sites to provide abasic cDNA;
reacting said abasic DNA with a primary amine bearing a detectable moiety having the formula Q-L-NH2, wherein Q is a detectable moiety and L is a linker to provide labeled DNA fragments;
hybridizing said labeled fragments to a nucleic acid array to provide a hybridization pattern; and analyzing the hybridization pattern.

According to the above method, the photocleavable nucleotide derivative is 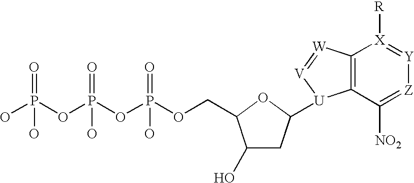
wherein U, V, W, X, Y and Z are C or N or any combination thereof, R is H, OH or NH₂, wherein if R is NH2, X is C and wherein if X is NH2, R is a pair of non-bonded electrons. More preferably, the photocleavable nucleotide derivative has the structure

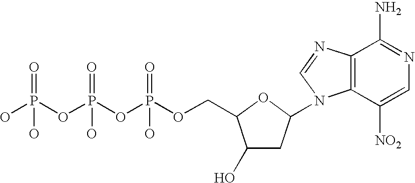

The photocleavable nucleotide derivative is preferably cleaved by light having a wavelength of from 320 nm up to approximately 380 nm. More preferably, the cleavage wavelength is about 365 nm.

Preferably, the fragments are generated having an average size range selected from the group consisting of 10, 20, 30, 40, 50, 60, 70, 80, 100 or 200 nucleotides. In accordance with instantly disclosed methods the cleaving and the labeling steps are preferably carried out simultaneous.

In accordance with the present invention, cDNA is preferably ss-cDNA. In another preferred embodiment of the instant invention, cDNA is preferably ds-cDNA.

In accordance with another preferred embodiment of the present invention, the photocleavable nucleotide is preferably 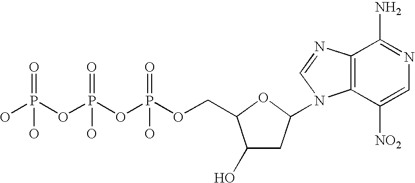
which is preferably incorporated into the ss-cDNA during reverse transcription. In another preferred embodiment of the instant invention, 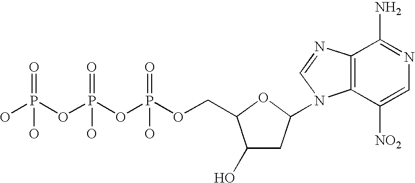
is incorporated into the ds-cDNA during second strand cDNA synthesis. According to yet another preferred embodiment of the instant invention, 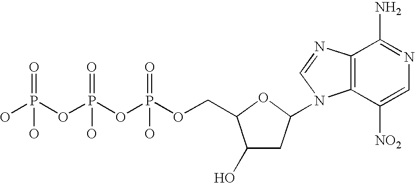
is incorporated in a single or in both strands of ds-cDNA.
Preferably Q is Biotin

In order to meet these requirements, persons of skill in the art can modify the enzyme to accept different substrates, for example by deleting or changing amino acids in the enzyme. In addition, substrates can be modified in a number of ways so that they work more efficiently and with greater fidelity with available wild type or mutant enzymes. Searching for variants in the enzymes and substrates to identify optimal combinations is within the ambit of those of skill in the art without undue experimentation.

All patents, patent applications, and literature cited in the specification are hereby incorporated by reference in their entirety. In the case of any inconsistencies, the present disclosure, including any definitions therein will prevail.

The invention has been described with reference to various specific and preferred embodiments and techniques. However, it should be understood that many variations and modifications may be made while remaining within the spirit and scope of the invention.

What is claimed is:

1. A method for analyzing a nucleic acid sample containing mRNA, said method comprising the following steps:
providing a nucleic acid sample containing mRNA;
synthesizing cDNA in the presence of a photocleavable nucleotide derivative, wherein said photocleavable nucleotide derivative provides abasic DNA upon incorporation into a DNA strand, following exposure to light of an appropriate wavelength;
exposing said cDNA to light of a predetermined wavelength to cause photocleavage and formation of a plurality of abasic sites to provide abasic cDNA;
cleaving said abasic cDNA with an endonuclease as to generate a plurality of fragments with terminal free 3′ hydroxyl groups;
labeling said fragments with biotin using terminal transferase;
hybridizing said labeled fragments to a nucleic acid array to provide a hybridization pattern; and
analyzing the hybridization pattern.

2. A method according to claim 1 wherein photocleavable nucleotide derivative is 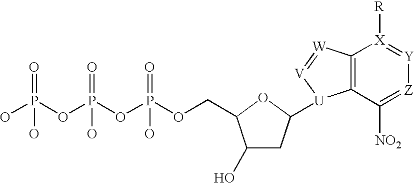
wherein U, V, W, X, Y and Z are C or N or any combination thereof, R is H, OH or NH₂, wherein if R is NH2, X is C and wherein if X is NH2, R is a pair of non-bonded electrons.

3. A method according to claim 2 wherein said photocleavable nucleotide derivative has the structure

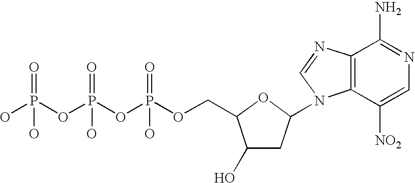

4. A method according to claim 2 wherein said light has a wavelength of from 320 nm up to approximately 380 nm.

5. A method according to claim 4 wherein said light has a wavelength of about 365 nm.

6. A method according to claim 1 wherein said endonuclease is endonuclease IV.

7. A method according to claim 1 wherein said endonuclease is endonuclease ApeI.

8. A method according to claim 1 wherein the cDNA is cleaved at abasic sites by endonuclease V.

9. A method according to claim 1 wherein fragments size range from at least 10 bps to 200 bps.

10. A method according to claim 1 wherein the cleaving and the labeling steps are carried out simultaneous.

11. A method according to claim 1 wherein the nucleic acid sample is mRNA.

12. A method according to claim 1 wherein the cDNA is ss-cDNA.

13. A method according to claim 1 wherein the cDNA is ds-cDNA.

14. A method according to claim 1 wherein 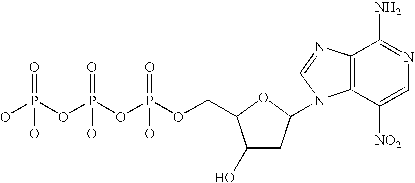
is incorporated into the ss-cDNA during reverse transcription.

15. A method according to claim 1 wherein 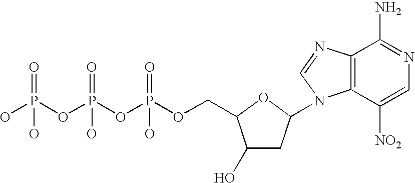
is incorporated into the ds-cDNA during second strand cDNA synthesis.

16. A method according to claim 15 wherein 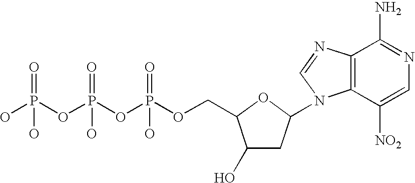
is incorporated in a single or in both strands of ds-cDNA.

17. A method for analyzing a nucleic acid sample containing RNA, said method comprising the following steps:
providing a nucleic acid sample containing RNA;
synthesizing cDNA in the presence of a photocleavable nucleotide derivative, wherein said photocleavable nucleotide derivative provides abasic DNA upon incorporation into a DNA strand, following exposure to light of an appropriate wavelength;
exposing said cDNA to light of a predetermined wavelength to cause photocleavage and formation of a plurality of abasic sites to provide abasic cDNA;
incubating said abasic DNA with in basic conditions to provide DNA fragments having 3′ terminal phosphate groups;
dephosphorylating said fragments to provide 3′ terminal OH groups; and labeling said fragments with biotin using terminal transferase;
hybridizing said labeled fragments to a nucleic acid array to provide a hybridization pattern; and
analyzing the hybridization pattern.

18. A method according to claim 17 wherein photocleavable nucleotide derivative is 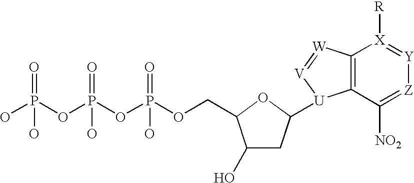
wherein U, V, W, X, Y and Z are C or N or any combination thereof, R is H, OH or NH₂, wherein if R is NH2, X is C and wherein if X is NH2, R is a pair of non-bonded electrons.

19. A method according to claim 18 wherein said photocleavable nucleotide derivative has the structure

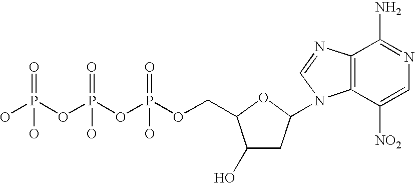

20. A method according to claim 18 wherein said light has a wavelength of from 320 nm up to approximately 380 nm.

21. A method according to claim 20 wherein said light has a wavelength of about 365 nm.

22. A method according to claim 18 wherein the nucleic acid sample is mRNA.

23. A method according to claim 18 wherein the cDNA is ss-cDNA.

24. A method according to claim 18 wherein the cDNA is ds-cDNA.

25. A method according to claim 18 wherein 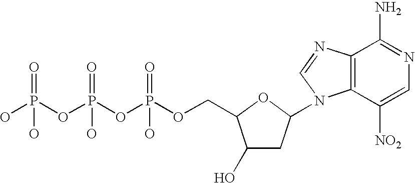
is incorporated into the ss-cDNA during reverse transcription.

26. A method according to claim 18 wherein 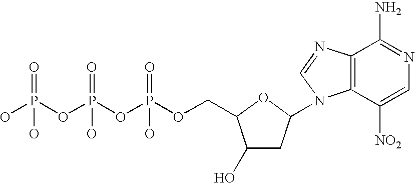
is incorporated into the ds-cDNA during second strand cDNA synthesis.

27. A method according to claim 18 wherein 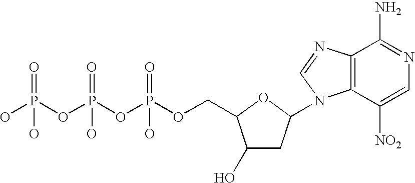
is incorporated in a single or in both strands of ds-cDNA.

28. A method for analyzing a nucleic acid sample containing RNA, said method comprising the following steps:
providing a nucleic acid sample containing RNA;
synthesizing cDNA in the presence of a photocleavable nucleotide derivative, wherein said photocleavable nucleotide derivative provides abasic DNA upon incorporation into a DNA strand, following exposure to light of an appropriate wavelength;
exposing said cDNA to light of a predetermined wavelength to cause photocleavage and formation of a plurality of abasic sites to provide abasic cDNA;
reacting said abasic DNA with a primary amine bearing a detectable moiety having the formula Q-L-NH2, wherein Q is a detectable moiety and L is a linker to provide labeled DNA fragments;
hybridizing said labeled fragments to a nucleic acid array to provide a hybridization pattern; and
analyzing the hybridization pattern.

29. A method according to claim 28 wherein photocleavable nucleotide derivative is 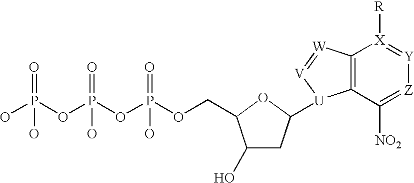
wherein U, V, W, X, Y and Z are C or N or any combination thereof, R is H, OH or NH₂, wherein if R is NH2, X is C and wherein if X is NH2, R is a pair of non-bonded electrons.

30. A method according to claim 29 wherein said photocleavable nucleotide derivative has the structure

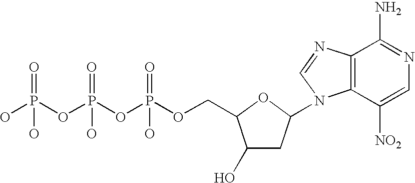

31. A method according to claim 28 wherein said light has a wavelength of from 320 nm up to approximately 380 nm.

32. A method according to claim 31 wherein said light has a wavelength of about 365 nm. A method according to claim 28 wherein fragments size range from at least 10 bps to 200 bps.

33. A method according to claim 28 wherein the nucleic acid sample is mRNA.

34. A method according to claim 28 wherein the cDNA is ss-cDNA.

35. A method according to claim 28 wherein the cDNA is ds-cDNA.

36. A method according to claim 28 wherein 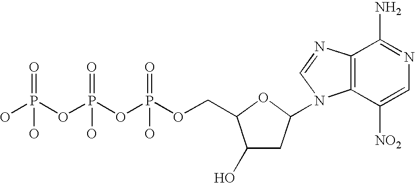
is incorporated into the ss-cDNA during reverse transcription.

37. A method according to claim 28 wherein 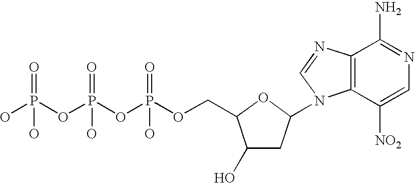
is incorporated into the ds-cDNA during second strand cDNA synthesis.

38. A method according to claim 28 wherein 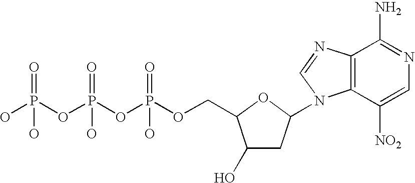
is incorporated in a single or in both strands of ds-cDNA.

39. A method according to claim 28 wherein Q is biotin.
